(19) United States
(12) Patent Application Publication
(10) Pub. No.: US 2021/0330594 A1
(43) Pub. Date: Oct. 28, 2021

(54) Fast-Acting Soft Gelatin Capsules Containing A Mixture Of Pain Reliever/Fever Reducer, Diuretic And Antihistamine

(71) Applicant: PuraCap Pharmaceutical LLC, Iselin, NJ (US)

(72) Inventor: Charchil Vejani, Mount Laurel, NJ (US)

(21) Appl. No.: 17/274,306

(22) PCT Filed: Sep. 10, 2019
(86) PCT No.: PCT/US19/50297

§ 371 Date: Mar. 8, 2021

Related U.S. Application Data
(60) Provisional application No. 62/729,737, filed on Sep. 11, 2018.

(51) Int. Cl. A61K 9/48; A61K 31/167; A61K 31/522; A61K 31/4402

(52) U.S. Cl. CPC A61K 9/4866; A61K 31/167; A61K 9/4833; A61K 31/4402; A61K 9/4858; A61K 31/522

(57) ABSTRACT

Fast-acting, bioavailable liquid softgel fill compositions comprising (a1) a pain reliever/fever reducer; (a2) a diuretic; (a3) an antihistamine; (b1) a pharmaceutically acceptable polyalkylene glycol; (b2) a pharmaceutically acceptable alkylene glycol; (c1) a pharmaceutically acceptable polymeric solubilizer; (c2) water; and (d) an antioxidant/preservative are disclosed, where the liquid softgel fill composition does not crystallize or otherwise deposit any of the active ingredients. Also disclosed are methods for the preparation of such fill compositions, and softgel capsules containing the bioavailable liquid fill composition.

### FIELD OF THE DISCLOSED SUBJECT MATTER

The presently disclosed subject matter relates to bioavailable fill compositions containing a pain reliever/fever reducer, diuretic and antihistamine, soft gelatin capsules filled with the bioavailable fill compositions to provide rapid action of the active ingredients, and methods of making same.

### BACKGROUND

Combinations of various medications are frequently taken by patients suffering from premenstrual syndrome (PMS). PMS is a term commonly used to describe physical and mental symptoms which occur from seven to fourteen days prior to the onset of the menstrual flow. Discomforts can range from primary dysmenorrhea, which includes painful menstruation and cramping, to headache, backache, irritability and bloating.

Women of menstruating age typically have menstrual cycles averaging 28 days, with some variation based on age, and physical and emotional wellbeing, as well as other factors. The duration of menstrual flow is variable, but typically lasts from three to seven days.

Symptoms of PMS range from mild to incapacitating and may involve some form of temporary mental or physical incapacitation. Psychological symptoms can include irritability, lethargy, depression, anxiety, sleep disorders, crying and hostility. Neurological symptoms can include headache, dizziness, fainting and seizures. Physical symptoms can include tenderness and swelling in the breasts, constipation, abdominal bloating, cramping, edema, urination issues and acne.

These physical, neurological and psychological symptoms of PMS are a major cause of discomfort to women, and cause substantial loss of time and efficiency in the workplace. Combination medicines in a unitary formulation are sought for their convenience in treating such symptoms.

Thus, there is a need to develop a stable liquid formulation combining all active pharmaceutical ingredients (APIs) in a highly concentrated solution suitable for producing a rapid-release and fast-acting compact dosage form.

### SUMMARY OF THE DISCLOSED SUBJECT MATTER

A new formulation that meets these criteria is disclosed herein which provides a desirable combination of active ingredients, particularly a combination of at least one pain reliever/fever reducer, at least one diuretic and at least one antihistamine in a unitary, rapid-acting liquid formulation. This formulation provides convenience to the patient and rapid onset of action for relief of pain and other symptoms associated with menstruation and PMS.

The pharmaceutical formulation of this invention contains Acetaminophen as the pain reliever/fever reducer, Pamabrom as a mild diuretic, and Pyrilamine Maleate as an antihistamine, in a solvent system which dissolves all three active pharmaceutical ingredients (APIs). A highly concentrated solution of the inventive combination allows a high dose of Acetaminophen (about 325 mg), about 25 mg of Pamabrom and about 15 mg of Pyrilamine Maleate to be formulated in a compact oral dosage form.

One aspect of the invention is directed to a concentrated bioavailable liquid softgel fill composition comprising (a1) acetaminophen as active ingredient; (a2) Pamabrom as active ingredient; (a3) a Pyrilamine salt as active ingredient; (b1) a pharmaceutically acceptable polyalkylene glycol; (b2) a pharmaceutically acceptable alkylene glycol; (c1) a pharmaceutically acceptable polymeric solubilizer; (c2) water; and (d) an antioxidant/preservative, where the liquid softgel fill composition does not crystallize or otherwise deposit any of the active ingredients. The bioavailable liquid softgel fill composition can comprise about 10 to about 50 wt % acetaminophen, about 0.3 to about 5 wt % Pamabrom, about 0.3 to about 5 wt % pyrilamine salt, about 30 to about 70 wt % polyalkylene glycol, about 0.3 to about 5 wt % alkylene glycol, about 1 to about 30 wt % polymeric solubilizer, about 1 to about 10 wt % water, and about 0.01 to about 1 wt % antioxidant/preservative, where the wt % values are based on the total weight of the composition. In one embodiment the bioavailable liquid softgel fill composition comprises about 25 wt % acetaminophen, about 2 wt % Pamabrom, about 1.2 wt % of a Pyrilamine salt, about 55 wt % of polyalkylene glycol, about 2 wt % alkylene glycol, about 8 wt % polymeric solubilizer, about 7 wt % water, and about 0.1 wt % of antioxidant/preservative, where the wt % values are based on the total weight of the composition. The Pyrilamine salt is preferably Pyrilamine Maleate. The polyalkylene glycol is preferably polyethylene glycol 400. The alkylene glycol is preferably propylene glycol. The polymeric solubilizer is a preferably a polyvinylpyrrolidone (PVP). The PVP is preferably PVP K17. The antioxidant/preservative is preferably butylated hydroxyanisole (BHA). In a preferred embodiment of the concentrated bioavailable liquid softgel fill composition, the Pyrilamine salt is Pyrilamine Maleate, the polyalkylene glycol is polyethylene glycol 400, the alkylene glycol is propylene glycol, the polymeric solubilizer is polyvinylpyrrolidone, and the antioxidant/preservative is BHA.

Another aspect of the invention is directed to a softgel capsule comprising a soft gelatin shell filled with the concentrated bioavailable liquid softgel fill composition as described above.

A further aspect of the invention is directed to a softgel capsule which comprises: a soft gelatin shell, and a concentrated liquid softgel fill composition within the shell, where the liquid softgel fill composition comprises: (a1) about 10 to about 50 wt % acetaminophen; (a2) about 0.3 to about 5 wt % Pamabrom; (a3) about 0.3 to about 5 wt % Pyrilamine salt; (b1) about 30 to about 70 wt % pharmaceutically acceptable polyalkylene glycol; (b2) about 0.3 to about 5 wt % pharmaceutically acceptable alkylene glycol; (c1) about 1 to about 30 wt % pharmaceutically acceptable polymeric solubilizer; (c2) about 1 to about 10 wt % water; and d) about 0.01 to about 1 wt % antioxidant/preservative; where the liquid softgel fill composition does not crystallize or otherwise deposit any of the active ingredients, and where the wt % values are based on the total weight of the composition. Preferably the Pyrilamine salt is Pyrilamine Maleate, the polyalkylene glycol is polyethylene glycol 400, the alkylene glycol is propylene glycol, the polymeric solubilizer is polyvinylpyrrolidone, and the antioxidant/preservative is BHA. Preferably the softgel capsule contains 325 mg of acetaminophen. Preferably the softgel capsule contains 25 mg of Pamabrom. Preferably the softgel capsule contains 15 mg of Pyrilamine Maleate. In a preferred embodiment, the softgel capsule contains 325 mg of acetaminophen, 25 mg of Pamabrom and 15 mg of Pyrilamine Maleate. Preferably the soft gelatin shell of the softgel capsule comprises about 30 to about 50 wt % gelatin, about 5 to about 20 wt % glycerin, about 5 to about 20 wt % sorbitol sorbitan solution, about 25 to about 35 wt % water, and optionally, pharmaceutically acceptable colorant.

Another aspect of the invention is directed to a method of preparing a concentrated liquid softgel fill composition, the method comprising: a) mixing polyethylene glycol, propylene glycol and water until a solution is obtained; b) adding BHA with mixing until completely dissolved; c) adding polyvinylpyrrolidone in portions with continuous mixing and heating to 50° C.±10° C. until dissolved; d) adding acetaminophen with continuous mixing and heating to 50° C.±10° C. until dissolved; e) adding Pamabrom with continuous mixing and heating to 50° C.±10° C. until dissolved; f) cooling the resulting fill solution to room temperature; g) adding Pyrilamine Maleate with continuous mixing until a clear solution is obtained; and h) optionally, deaerating the clear concentrated liquid softgel fill solution under vacuum.

### DETAILED DESCRIPTION

Without wishing to be bound by any particular theory, it is believed that oral absorption is possible only when an active pharmaceutical ingredient (API) is in a solution form or solubilized form in the gastrointestinal tract (GIT), allowing the API to diffuse into and across the enterocytes lining of the intestinal lumen. If the compound is administered as a solution or is otherwise solubilized, it eliminates any dissolution rate-limiting step in the absorption process. This yields a faster, more uniform and enhanced absorption.

If the API demonstrates sufficient solubility in a pharmaceutically acceptable nonaqueous vehicle, soft gelatin capsules are ideal to deliver the solution as a “solid” dosage form. However, maintaining stability of the compound in solution form over an extended shelf life period can be challenging, especially when two or more active ingredients are combined.

Over the counter active ingredients such as Acetaminophen, Pamabrom and Pyrilamine Maleate are used in various combinations to treat the symptoms of premenstrual syndrome (PMS), such as tension, bloating, water weight gain, headache, muscle pain, cramps, and irritability. However, combining three active ingredients in solution form and then encapsulating the solution in a softgel was anticipated to be challenging in terms of stability as well as the manufacturing process.

It has now been discovered that a highly concentrated solution of a combination of a high dose of acetaminophen, 325 mg, Pamabrom, 25 mg, and Pyrilamine Maleate, 15 mg can be stably formulated in a compact oral dosage form. Such a formulation also enhances the bioavailability of acetaminophen. However, acetaminophen tends to degrade or recrystallize in such a solution. Further, there is the possibility of interaction between the various active ingredients so that stability would be further compromised.

The inventive solvent system is a combination of nonaqueous solvents, co-solvents, crystallization inhibiting agents, antioxidants and optionally other adjuvants. Polyethylene glycol of different molecular weights is a commonly used nonaqueous and pharmaceutically acceptable solvent. It can be combined with a co-solvent such as propylene glycol to increase solubility of the above APIs. Crystallization inhibitors are used to prevent crystallization of active compounds from solution. Hydrophilic polymers, such as polyvinylpyrrolidone or cellulose polymers, are commonly used crystallization inhibitors. Such agents increase the physical distance between particles of active ingredient and reduce particle-particle interactions.

Polyethylene glycol, also known as “PEG,” has the formula H(OCH2CH2)nOH, wherein n is 4 or greater. A number generally follows the designation “PEG” to indicate its average molecular weight, e.g., PEG 400 or PEG 600. Polyethylene glycol is a clear viscous liquid or semisolid at room temperature, and is miscible with water and many organic solvents. Its molecular weight can be between 200 and 800, preferably 400 to 600, more preferably about 400. The solvent system can contain a single polyethylene glycol or a mixture of two or more polyethylene glycols.

Propylene glycol is a clear viscous liquid and has the formula HOCH2CH(OH)CH3. It is miscible with water and can be optionally included as co-solvent as a part of the solvent system described above.

Polyvinylpyrrolidone (PVP), also known as POVIDONE®, is a water-soluble polymeric crystallization inhibitor. The polyvinylpyrrolidone used herein preferably has a molecular weight in the range 2000 to 1500000, e.g., 2000 to 62000, 2000 to 4000, 4000 to 18000, or 6000 to 15000. Commercial polyvinylpyrrolidone products are commonly graded by K values. The K value is an index for correlating relative viscosity with the average degree of polymerization. The PVP used herein is selected from the group consisting of PVP K12, PVP K17, PVP K30, PVP K60, and PVP K90; preferably the polyvinylpyrrolidone is PVP 12, PVP 17 or PVP 30. More preferably the PVP is PVP 17. Polyvinylpyrrolidone enhances the solubility of APIs in the solvent system containing polyvinylpyrrolidone, polyethylene glycol, propylene glycol, water, and optionally other components, by preventing or inhibiting crystallization of the APIs.

Antioxidants are pharmaceutical excipients that prevent or delay oxidative degradation of active ingredients. Antioxidants are generally classified into three groups as: phenolic antioxidants (sometimes called true antioxidants), reducing agents, and chelating agents. Phenolic antioxidants are sterically hindered phenols that react with free radicals, thereby blocking any radical-induced chain reaction. These hindered phenols serve as radical-trapping antioxidants for oxy- and peroxy-radicals. The resulting phenoxy radicals that form are stabilized by steric hindrance of their bulky substituents and thus cannot attach drugs or unsaturated fatty acids to maintain a chain reaction. The hindered phenoxy radical is stabilized by delocalization of the unpaired electron around the phenol ring to form a stable resonance hybrid. Butylated hydroxytoluene (BHT), propyl gallate and butylated hydroxyanisole (BHA) are examples of synthetic hindered phenols useful as antioxidants in the inventive formulations. An example of a natural hindered phenol useful as an antioxidant is α-tocopherol.

Reducing antioxidants are compounds that have lower redox potentials, and thus are more readily oxidized than the drug they are intended to protect. Reducing agents scavenge oxygen from the medium, and thus delay or prevent drug oxidation.

Chelating antioxidants are sometimes called antioxidant synergists. Metal ions, such as Co2+, Cu2+, Fe3+, Fe2+ and Mn2+ shorten a radical induction period and increase the oxidation rate. Trace amounts of these metal ions are frequently unintentionally introduced into drug products during manufacturing. Chelating agents do not possess antioxidant activity as such, but enhance the action of phenolic antioxidants by reacting with (chelating) any catalyzing metal ions present, forming chelates that are inactive with regard to radical chain propagation.

One aspect of the invention is directed to a concentrated bioavailable liquid softgel fill composition comprising (a1) a pain reliever/fever reducer as active ingredient; (a2) a diuretic as active ingredient; (a3) an antihistamine as active ingredient; (b1) a pharmaceutically acceptable polyalkylene glycol; (b2) a pharmaceutically acceptable alkylene glycol; (c1) a pharmaceutically acceptable polymeric solubilizer; (c2) water; and (d) an antioxidant/preservative, where the liquid softgel fill composition does not crystallize or otherwise deposit any of the active ingredients.

Another aspect of the invention is directed to a concentrated bioavailable liquid softgel fill composition comprising (a1) acetaminophen as the pain reliever/fever reducer; (a2) Pamabrom as the diuretic; (a3) a Pyrilamine salt as the antihistamine; (b1) a pharmaceutically acceptable polyalkylene glycol; (b2) a pharmaceutically acceptable alkylene glycol; (c1) a pharmaceutically acceptable polymeric solubilizer; (c2) water; and (d) an antioxidant/preservative, where the liquid softgel fill composition does not crystallize or otherwise deposit any of the active ingredients.

The bioavailable liquid softgel fill composition can comprise about 10 to about 50 wt % acetaminophen, about 0.3 to about 5 wt % Pamabrom, about 0.3 to about 5 wt % Pyrilamine salt, about 30 to about 70 wt % polyalkylene glycol, about 0.3 to about 5 wt % alkylene glycol, about 1 to about 30 wt % polymeric solubilizer, about 1 to about 10 wt % water, and about 0.01 to about 1 wt % antioxidant/preservative, where the wt % values are based on the total weight of the composition. The acetaminophen content can be about 10 to about 50, or about 15 to about 40, or about 20 to about 30 wt %. The acetaminophen content can be about 10, 15, 20, 25, 30, 35, 40, 45, or about 50 wt %. The Pamabrom content can be about 0.3 to about 5, or about 0.5 to about 4, or about 0.75 to about 3, or about 1 to about 2.5 wt %. The Pamabrom content can be about 0.3, 0.5, 0.7, 0.9, 1, 1.2, 1.4, 1.6, 1.8, 1.9, 2.0, 2.2, 2.5, 3, 3.5, 4, 4.5 or about 5 wt %. The Pyrilamine salt content can be about 0.3 to about 5, or about 0.5 to about 4, or about 0.7 to about 3, or about 0.9 to about 2.5, or about 1 to about 2 wt %. The Pyrilamine salt content can be about 0.3, 0.5, 0.7, 0.9, 1, 1.1, 1.2, 1.3, 1.4, 1.5, 1.6, 1.8, 2.0, 2.2, 2.5, 3, 3.5, 4, 4.5 or about 5 wt %. The polyalkylene glycol content can be about 30 to 70, or about 35 to 65, or about 40 to 60, or about 45 to 55 wt %. The polyalkylene glycol content can be about 30, 35, 40, 45, 50, 55, 60, 65, or about 70 wt %. The alkylene glycol can be about 0.3 to about 5, or about 0.5 to about 4.5, or about 0.75 to about 4, or about 1 to about 3.5, or about 1.5 to about 3, or about 1.75 to about 2.5, or about 2 to about 2.25 wt %. The alkylene glycol can be about 0.3, 0.5, 0.75, 1, 1.5, 1.75, 2, 2.25, 2.5, 3, 3.5, 4, 4.5, or about 5 wt %. The polymeric solubilizer can be present in about 1 to about 30, or about 2 to about 20, or about 3 to about 15, or about 4 to about 10, or about 5 to about 9, or about 6 to about 8.5, or about 7 to about 8 wt %. The polymeric solubilizer can be present in about 1, 2, 3, 4, 5, 6, 7, 7.5, 7.75, 8, 9, 10, 12, 14, 16, 18, 10, 22, 24, 26, 28, or about 30 wt %. The water can be present in about 1 to about 10, or about 2 to about 9, or about 4 to about 8, or about 5 to about 7.5 wt %. The water can be present in about 1, 2, 3, 4, 5, 6, 6.25, 6.5, 6.75, 7, 7.5, 7.75, 8, 8.5, 9, 9.5, or about 10 wt %. The antioxidant/preservative can be present in about 0.01 to about 1, or about 0.03 to about 0.095, or about 0.05 to about 0.9, or about 0.07 to about 0.85 wt %. The antioxidant/preservative can be present in about 0.01, 0.02, 0.03, 0.04, 0.05, 0.06, 0.07, 0.08, 0.09, 0.095, or about 1 wt %.

In one embodiment the bioavailable liquid softgel fill composition comprises about 25 wt % acetaminophen, about 2 wt % Pamabrom, about 1.2 wt % of a Pyrilamine salt, about 55 wt % of polyalkylene glycol, about 2 wt % alkylene glycol, about 8 wt % polymeric solubilizer, about 7 wt % water, and about 0.1 wt % of antioxidant/preservative, where the wt % values are based on the total weight of the composition. The Pyrilamine salt is preferably Pyrilamine Maleate. Other salts, such as the hydrohalides (e.g., HCl salt) are also suitable. The polyalkylene glycol is preferably a polyethylene glycol (PEG). Preferably the PEGs are selected from the group consisting of PEG 200, 300, 400, 600, mixtures thereof, and mixtures of these with PEG 800, 1000, 2000, 3000, 4000, 5000, 6000, 7000, or 8000. More preferably the polyalkylene glycol is PEG 400. The alkylene glycol is preferably propylene glycol. The polymeric solubilizer is a preferably a polyvinylpyrrolidone (PVP). The PVP is selected from the group consisting of PVP K12, PVP K17, PVP K30, PVP K60, and PVP K90; preferably the polyvinylpyrrolidone is PVP 12, PVP 17 or PVP 30. More preferably the PVP is PVP K17. The antioxidant/preservative is preferably selected from butylated hydroxytoluene (BHT), propyl gallate and butylated hydroxyanisole (BHA). More preferably the antioxidant/preservative is butylated hydroxyanisole (BHA). In a preferred embodiment of the concentrated bioavailable liquid softgel fill composition, the Pyrilamine salt is Pyrilamine Maleate, the polyalkylene glycol is polyethylene glycol 400, the alkylene glycol is propylene glycol, the polymeric solubilizer is polyvinylpyrrolidone, and the antioxidant/preservative is BHA.

In one embodiment the bioavailable liquid softgel fill composition contains 25.00 wt % acetaminophen, 1.92 wt % Pamabrom, 1.15 wt % Pyrilamine Maleate, 55.46 wt % polyethylene glycol 400, 2.15 wt % propylene glycol, 7.69 wt % polyvinylpyrrolidone K17 (PVP K17), 6.54 wt % water, and 0.08 wt % of butylated bydroxyanisole (BHA), where the wt % values are based on the total weight of the composition.

Another aspect of the invention is directed to a softgel capsule comprising a soft gelatin shell filled with the concentrated bioavailable liquid softgel fill composition as described above. In one embodiment the softgel capsule shell contains 43.30 wt % gelatin, 10.94 wt % glycerin, 13.71 wt % sorbitol sorbitan solution, and 32.15 wt % water, where the wt % values are based on the total weight of the softgel capsule shell. The gelatin of the soft gelatin (softgel) capsule can comprise bovine-, avian-, porcine-, marine- or vegetable-based gelatin, or a mixture of two or more thereof. The softgel capsule can further comprise an enteric coating. The enteric coating preferably comprises a controlled release or delayed release polymer. The controlled release polymer can be an acid-resistant polymer.

The liquid softgel fill formulation can be encapsulated in soft gelatin shells to form softgel capsules using a conventional rotary die process. Suitable soft gelatin shells can include (i) gelatin, 35-60% by weight; (ii) glycerin, 0-15% by weight; (iii) sorbitol/sorbitan, 0-20% by weight; (iv) purified water, 20-40% by weight; and (v) artificial color, 0.0001-0.002% by weight.

The softgel capsules of the invention can also be prepared by other methods well known in the art. See e.g., P. K. Wilkinson et al., “Softgels: Manufacturing Considerations,” Drugs and the Pharmaceutical Sciences, 41 (Specialized Drug Delivery Systems); P. Tyle, Ed. (Marcel Dekker, Inc., New York, 1990) 409-449; F. S. Hom et al., “Capsules, Soft” Encyclopedia of Pharmaceutical Technology, vol. 2; J. Swarbrick and J. C. Boylan, eds. (Marcel Dekker, Inc., New York, 1990) pp. 269-284; M. S. Patel et al., “Advances in Softgel Formulation Technology,” Manufacturing Chemist, vol. 60, no. 7, pp. 26-28 (July 1989); M. S. Patel et al., “Softgel Technology,” Manufacturing Chemist, vol. 60, no. 8, pp. 47-49 (August 1989); R. F. Emerson, “Softgel (Soft Gelatin Capsule) Update,” Drug Development and Industrial Pharmacy (Interphex '86 Conference), vol. 12, no. 8 & 9, pp. 1133-1144 (1986); and W. R. Ebert, “Soft Elastic Gelatin Capsules: A Unique Dosage Form,” Pharmaceutical Technology, vol. 1, no. 5, pp. 44-50 (1977).

A further aspect of the invention is directed to a softgel capsule which comprises: a soft gelatin shell, and a concentrated liquid softgel fill composition within the shell, where the liquid softgel fill composition comprises: (a1) about 10 to about 50 wt % acetaminophen; (a2) about 0.3 to about 5 wt % Pamabrom; (a3) about 0.3 to about 5 wt % Pyrilamine salt; (b1) about 30 to about 70 wt % pharmaceutically acceptable polyalkylene glycol; (b2) about 0.3 to about 5 wt % pharmaceutically acceptable alkylene glycol; (c1) about 1 to about 30 wt % pharmaceutically acceptable polymeric solubilizer; (c2) about 1 to about 10 wt % water; and d) about 0.01 to about 1 wt % antioxidant/preservative; where the liquid softgel fill composition does not crystallize or otherwise deposit any of the active ingredients, and where the wt % values are based on the total weight of the composition. Preferably the Pyrilamine salt is Pyrilamine Maleate, the polyalkylene glycol is polyethylene glycol 400, the alkylene glycol is propylene glycol, the polymeric solubilizer is polyvinylpyrrolidone, and the antioxidant/preservative is BHA. Preferably the softgel capsule contains 325 mg of acetaminophen. Preferably the softgel capsule contains 25 mg of Pamabrom. Preferably the softgel capsule contains 15 mg of Pyrilamine Maleate. In a preferred embodiment, the softgel capsule contains 325 mg of acetaminophen, 25 mg of Pamabrom and 15 mg of Pyrilamine Maleate. Preferably the soft gelatin shell of the softgel capsule comprises about 30 to about 50 wt % gelatin, about 5 to about 20 wt % glycerin, about 5 to about 20 wt % sorbitol sorbitan solution, about 25 to about 35 wt % water, and optionally, pharmaceutically acceptable colorant.

Another aspect of the invention is directed to a method of preparing a concentrated liquid softgel fill composition, the method comprising: a) mixing about 30 to about 70 wt % polyalkylene glycol, about 0.3 to about 5 wt % alkylene glycol, and about 1 to about 10 wt % water until a solution is obtained; b) adding about 0.01 to about 1 wt % antioxidant/preservative, such as BHA, BHT or propyl gallate, with mixing until completely dissolved; c) adding about 1 to about 30 wt % pharmaceutically acceptable polymeric solubilizer, such as PVP (polyvinylpyrrolidone), in portions with continuous mixing and heating to 50° C.±10° C. until dissolved; d) adding acetaminophen with continuous mixing and heating to 50° C.±10° C. until dissolved; e) adding Pamabrom with continuous mixing and heating to 50° C.±10° C. until dissolved; f) cooling the resulting fill solution to room temperature; g) adding a Pyrilamine salt, such as Pyrilamine Maleate, with continuous mixing until a clear solution is obtained; and h) optionally, deaerating the clear concentrated liquid softgel fill solution under vacuum.

In one embodiment the method comprises: a) mixing polyethylene glycol, propylene glycol and water until a solution is obtained; b) adding BHA with mixing until completely dissolved; c) adding polyvinylpyrrolidone in portions with continuous mixing and heating to 50° C.±10° C. until dissolved; d) adding acetaminophen with continuous mixing and heating to 50° C.±10° C. until dissolved; e) adding Pamabrom with continuous mixing and heating to 50° C.±10° C. until dissolved; f) cooling the resulting fill solution to room temperature; g) adding Pyrilamine Maleate with continuous mixing until a clear solution is obtained; and h) optionally, deaerating the clear concentrated liquid softgel fill solution under vacuum.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally includes up to plus or minus 10% of the indicated number. For example, “about 10%” can indicate a range of 9% to 11%, and “about 20” can mean from 18-22. Preferably, “about” includes up to plus or minus 6% of the indicated value. Alternatively “about” includes up to plus or minus 5% of the indicated value. Other meanings of “about” may be apparent from the context, such as rounding off, so, for example “about 1” can also mean from 0.5 to 1.4.

### EXAMPLES

### Example 1. Bioavailable Fill Composition with Acetaminophen, Pamabrom and Pyrilamine Maleate as Active Ingredients

| | | |
|---|---|---|
| Ingredients | % w/w | Function |

Fill Formulation

| | | |
|---|---|---|
| Polyethylene Glycol 400, NF | 55.46 | Solvent |
| Propylene Glycol, NF | 2.15 | Co-solvent |
| Polyvinylpyrrolidone K17, | 7.69 | Crystallization Inhibitor |
| NF (PVP K17) | | |
| Purified Water, USP | 6.54 | Solvent |
| Acetaminophen, USP | 25.00 | Pain Reliever/Fever Reducer |
| Pamabrom, USP | 1.92 | Mild Diuretic |
| Pyrilamine Maleate, USP | 1.15 | Antihistamine |
| Butylated Hydroxyanisole, | 0.08 | Antioxidant |
| NF (BHA) | | |
| Total | 100 | |

Shell Ingredients

| | | |
|---|---|---|
| Gelatin | 43.20 | Capsule Shell Base |
| Glycerin | 10.94 | Plasticizer |
| Sorbitol Sorbitan Solution* | 13.71 | Plasticizer |
| Purified Water, USP | 32.15 | Solvent |
| FD&C Yellow No. 6 | trace | Colorant |
| FD&C Red No. 40 | trace | Colorant |
| Total | 100 | |

*Sorbitol Sorbitan Solution is a water solution containing, on the anhydrous basis, not less than 25.0% of D-sorbitol (C₆H₁₄O₆) and not less than 15.0% of 1,4-sorbitan (C₆H₁₂O₅).

All ingredients were mixed according to the procedure of Example 2.

### Example 2. Process for Preparation of Fill Composition of Example 1

1. Weigh accurately Polyethylene Glycol 400 and transfer into a suitable container.
2. Weigh accurately Propylene Glycol and transfer into Step 1. Begin moderate mixing.
3. Weigh accurately purified water and add into step 2. Continue mixing at moderate speed.
4. Weigh accurately BHA and transfer into step 3. Mix until it completely dissolves.
5. Weigh accurately PVP K-17 and add into step 4. Continue mixing at moderate speed till it completely dissolves. Heat the solution to about 50° C. Record speed, temperature and total mixing time.
6. Weigh accurately Acetaminophen, USP and transfer into step 5. Mix until it completely dissolves. Record speed, temperature and total mixing time.
7. Weigh accurately Pamabrom, USP and transfer into step 6. Mix until it completely dissolves. Record speed, temperature and total mixing time.
8. Stop heating and allow fill solution to cool to room temperature.
9. Weigh Pyrilamine Maleate, USP and transfer into cooled step 8. Continue mixing until it forms a clear solution.
10. If degasification is needed, while keeping the mixer at slow speed, place the fill preparation container under a vacuum deaerator. Turn on the vacuum pump and mix while maintaining a vacuum between −45 to −76 cm Hg on the vacuum gauge for 5 to 30 minutes. No visible bubbles should present after deaeration.

The above fill solution was encapsulated into a soft gelatin capsule (softgel) having a volume of 1.15 mL, which accommodates the 1153 mg of fill solution. Optionally, the softgel capsules are subsequently provided with an enteric coating consisting of hydroxypropyl methyl cellulose stearate and castor oil as plasticizer, in the customary manner.

### Example 3. Stability Determination

Manufactured softgels are evaluated to predict shelf life of the product. Softgel samples are kept under accelerated conditions (ACC) of 40° C./75% RH for 180 days. Samples are withdrawn and tested at predetermined periods for API assay and homogeneity. Results are compared against the label specification; if ACC-6M results (i.e., at 6 months) are within specification then a product shelf life of 2 years is predicted.

| | | | Assay % of label | | |
|---|---|---|---|---|---|
| | Label | T₀ % | 40° C./75% RH | | |
| API | (mg) | of label | 1 M | 2 M | 3 M |
| Acetaminophen | 325 | 94.2 | 94.0 | 94.1 | 92.2 |
| Pamabrom | 25 | 95.5 | 95.1 | 95.0 | 93.4 |
| Pyrilamine Maleate | 15 | 94.3 | 94.0 | 94.2 | 93.9 |

The specific examples disclosed above are to be construed as merely illustrative, and not limitative of the remainder of the disclosure in any way whatsoever. Without further elaboration, and based on the description herein, it is believed that one skilled in the art can utilize the present invention to its fullest extent.

### OTHER EMBODIMENTS

All of the features disclosed in this specification can be combined in any combination. Each feature disclosed in this specification can be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the present claims.

All publications cited herein are hereby incorporated by reference in their entirety.

What is claimed is:

1. A concentrated bioavailable liquid softgel fill composition comprising:
(a1) acetaminophen as active ingredient;
(a2) Pamabrom as active ingredient;
(a3) a Pyrilamine salt as active ingredient;
(b1) a pharmaceutically acceptable polyalkylene glycol;
(b2) a pharmaceutically acceptable alkylene glycol;
(c1) a pharmaceutically acceptable polymeric solubilizer;
(c2) water; and
(d) an antioxidant/preservative;
wherein said liquid softgel fill composition does not crystallize or deposit any of the active ingredients.

2. The bioavailable liquid softgel fill composition of claim 1, comprising about 10 to about 50 wt % acetaminophen, about 0.3 to about 5 wt % Pamabrom, about 0.3 to about 5 wt % pyrilamine salt, about 30 to about 70 wt % polyalkylene glycol, about 0.3 to about 5 wt % alkylene glycol, about 1 to about 30 wt % polymeric solubilizer, about 1 to about 10 wt % water, and about 0.01 to about 1 wt % antioxidant/preservative, wherein said wt % values are based on the total weight of the composition.

3. The bioavailable liquid softgel fill composition of claim 1, comprising about 25 wt % acetaminophen, about 2 wt % Pamabrom, about 1.2 wt % of a Pyrilamine salt, about 55 wt % of polyalkylene glycol, about 2 wt % alkylene glycol, about 8 wt % polymeric solubilizer, about 7 wt % water, and about 0.1 wt % of antioxidant/preservative, wherein said wt % values are based on the total weight of the composition.

4. The bioavailable liquid softgel fill composition of claim 1, wherein said Pyrilamine salt is Pyrilamine Maleate.

5. The bioavailable liquid softgel fill composition of claim 1, wherein said polyalkylene glycol is polyethylene glycol 400.

6. The bioavailable liquid softgel fill composition of claim 1, wherein said alkylene glycol is propylene glycol.

7. The bioavailable liquid softgel fill composition of claim 1, wherein said polymeric solubilizer is a polyvinylpyrrolidone.

8. The bioavailable liquid softgel fill composition of claim 7, wherein said polyvinylpyrrolidone is polyvinylpyrrolidone K17.

9. The bioavailable liquid softgel fill composition of claim 1, wherein said antioxidant/preservative is butylated hydroxyanisole (BHA).

10. The bioavailable liquid softgel fill composition of claim 1, wherein said Pyrilamine salt is Pyrilamine Maleate, said polyalkylene glycol is polyethylene glycol 400, said alkylene glycol is propylene glycol, said polymeric solubilizer is polyvinylpyrrolidone, and said antioxidant/preservative is BHA.

11. A softgel capsule comprising a soft gelatin shell filled with the concentrated bioavailable liquid softgel fill composition of claim 1.

12. A softgel capsule which comprises:
a soft gelatin shell, and
a concentrated liquid softgel fill composition within the shell, said liquid softgel fill composition comprising:
(a1) about 10 to about 50 wt % acetaminophen;
(a2) about 0.3 to about 5 wt % Pamabrom;
(a3) about 0.3 to about 5 wt % Pyrilamine salt;
(b1) about 30 to about 70 wt % pharmaceutically acceptable polyalkylene glycol;
(b2) about 0.3 to about 5 wt % pharmaceutically acceptable alkylene glycol;
(c1) about 1 to about 30 wt % pharmaceutically acceptable polymeric solubilizer;
(c2) about 1 to about 10 wt % water; and
(d) about 0.01 to about 1 wt % antioxidant/preservative;
wherein said liquid softgel fill composition does not crystallize or deposit any of the active ingredients, and wherein said wt % values are based on the total weight of the composition.

13. The softgel capsule of claim 12, wherein for said fill composition said Pyrilamine salt is Pyrilamine Maleate, said polyalkylene glycol is polyethylene glycol 400, said alkylene glycol is propylene glycol, said polymeric solubilizer is polyvinylpyrrolidone, and said antioxidant/preservative is BHA.

14. The softgel capsule of claim 12, wherein the total amount of acetaminophen is 325 mg.

15. The softgel capsule of claim 12, wherein the total amount of Pamabrom is 25 mg.

16. The softgel capsule of claim 12, wherein the Pyrilamine salt is Pyrilamine Maleate, present in a total amount of 15 mg.

17. The softgel capsule of claim 12, wherein the total amount of acetaminophen is 325 mg, the total amount of Pamabrom is 25 mg, and the Pyrilamine salt is Pyrilamine Maleate, which is present in a total amount of 15 mg.

18. The softgel capsule of claim 17, wherein said soft gelatin shell comprises about 30 to about 50 wt % gelatin, about 5 to about 20 wt % glycerin, about 5 to about 20 wt % sorbitol sorbitan solution, about 25 to about 35 wt % water, and optionally, pharmaceutically acceptable colorant.

19. The softgel capsule of claim 11, wherein said soft gelatin shell comprises about 30 to about 50 wt % gelatin, about 5 to about 20 wt % glycerin, about 5 to about 20 wt % sorbitol sorbitan solution, about 25 to about 35 wt % water, and optionally, pharmaceutically acceptable colorant.

20. A method of preparing a concentrated liquid softgel fill composition of claim 1, which method comprises:
a) Mixing polyethylene glycol, propylene glycol and water until a solution is obtained;
b) Adding BHA with mixing until completely dissolved;
c) Adding polyvinylpyrrolidone in portions with continuous mixing and heating to 50° C.±10° C. until dissolved;
d) Adding acetaminophen with continuous mixing and heating to 50° C.±10° C. until dissolved;
e) Adding Pamabrom with continuous mixing and heating to 50° C.±10° C. until dissolved;
f) Cooling fill solution to room temperature;
g) Adding Pyrilamine Maleate with continuous mixing until a clear solution is obtained; and
h) Optionally, deaerating the clear concentrated liquid softgel fill solution under vacuum.
